(19) United States
(12) Patent Application Publication
(10) Pub. No.: US 2024/0381835 A1
(43) Pub. Date: Nov. 21, 2024

(54) SMART CAGE SYSTEM AND METHOD FOR HOUSING AND ASSAYING MULTIPLE VERTEBRATE ANIMALS

(71) Applicant: Olden Labs, PBC., Allston, MA (US)

(72) Inventors: Michael Florea, Allston, MA (US); Noah Weber, Vienna (AT); Pablo Andres Penso Granier, Merida (VE)

(21) Appl. No.: 18/318,444

(22) Filed: May 16, 2023

(51) Int. Cl. A01K 1/03; A01K 5/00; A01K 7/00; A61B 5/00; A61B 5/11

(52) U.S. Cl. CPC A01K 1/031; A01K 5/00; A01K 7/00; A61B 5/1126; A61B 5/6887; A61B 2503/40

(57) ABSTRACT

Disclosed is a smart cage system for housing and assaying multiple vertebrate animals having at least one inner and outer housing assembly including at least one controller designed to monitor and record data from sensors, the at least one or more sensors operationally synchronized at least one or more of before, in real time, and after sensing an action, wherein data captured by each at least one or more sensors can be synchronized by way of at least one time measuring device. A multi object tracking software system is operationally coupled to at least one optical sensor by the at least one controller, designed to track individuals of the multiple vertebrate animals. At least the outer housing assembly includes at least one or more from a group of ports, slots, shelves, pockets, hooks, fasteners, and sleeves each designed to retain sensors and other elements.

### FIELD OF THE INVENTION

The inventive concept relates generally to a smart cage system and method for housing and assaying multiple vertebrate animals including at least one inner housing assembly, at least one outer housing assembly, and a multi object detection system.

### BACKGROUND

The longevity market in healthcare has undergone exceptional growth in therapeutics development, and most of researchers perform some form of longevity studies in rodents, particularly mice—the most popular mammalian aging model. In longevity studies, the prime metrics of interests are various physiological parameters that correlate with overall health (such as activity, strength, run endurance, spatial memory, coat density, and others) and lifespan of animals. Currently however, rodent longevity studies are conducted in mostly the same way as in the 1960s: animal rearing, health assays, and treatments are conducted manually, requiring a workforce and labor commensurate with the number of rodents in each study. Furthermore, because the natural biological variability of aging is high and effect sizes usually modest, the average longevity study uses about one hundred animals, with more recommended if possible to achieve statistical power for most assays. A single mouse longevity study, therefore, in the least expensive rodent model (mouse C57BL6/J) currently costs around $380,000-$600,000—to perform an 18-month long study measuring 10 different physiological parameters, for one intervention at one dose. The cost and time required for each test creates an incredible bottleneck and an obstacle for new longevity companies to overcome and may be one of the chief roadblocks that retards development in space today.

In effect, there are three types of providers relevant to the problem of mice longevity studies: 1) companies providing automated animal tracking, 2) general Contract Research Organizations (CROs), and 3) CROs providing aging studies. For the first of the three, three types of systems are typically used:
1. systems allowing tracking of invertebrates (Nemalife, Nagi Bio)
2. systems allowing RFID-based activity tracking of multiple mice (UiD, Kent Scientific)
3. “Smart cages” allowing short-term (up to a few weeks) automated multi-modal assaying of individual mice (Sable Biosystems, Vium)

However, none of these systems are sufficient to address the problem of mouse longevity testing. Tracking activity alone is insufficient, and smart cages designed to measure aspects of rodent health are not designed for long-term studies requiring high number of animals. As such, most who outsource their longevity studies use CROs experienced with longevity studies (Charles River, Ichor, Wuxi). These CROs perform the required assays manually using a team of animal technicians.

On longevity smart cages: because aging affects all systems of the body, and any given intervention may only improve health in a subset of systems, it is imperative to measure multiple different health parameters to draw useful inferences about the effects of a longevity intervention. All things being otherwise equal, the more assays that can be performed without affecting the health of the test subject, such as mice, the better. However, the usually small effect sizes and high variability of aging dictate that about one hundred animals are required to draw conclusions about the effect and effectiveness of an intervention.

Because of this large requirement for many test animals, performing health assays for even a single cohort of animals requires considerable human labor. For example, a health span study that required eight assays at two-time points for such measures as activity, frailty, muscle strength, memory, learning, coordination, endurance, vision also requires acclimatization of mice to the test room; training mice to perform the assay, and repeating measurements over several different days to arrive at an accurate value. Manual analysis of data (such as for video recordings), may also be required. Conducting one assay on one hundred mice, therefore, can easily take a whole week (40 person-hours). Multiplied by eight assays at two time points, means a conservative estimate of 640 man-hours per study in the representative example, usually far higher considering additional time required for adjourning necessary activities (such as moving cages and cleaning test setups) and data analysis. Labor intensity can result in a high cost per study than might be available through automation. However, more importantly, manual assays impose a limitation on the number of studies that can be run concurrently, as the amount of skilled labor available to do the studies is limited. Therefore, labor availability can be a greater limiter than cost. Furthermore, because manual assaying of mice requires mice to be handled and removed from their home environment, every additional assay increases stress and affects mouse behavior, both of which can change lifespan measurement results, probably negatively.

Therefore, many investigators opt to have separate cohorts of mice for health assays and lifespan measurement, further increasing the number of required mice. Yet, theoretically, using properly equipped monitoring cages, it is possible to measure health parameters directly in the home cage. Given purpose-designed cage technology, activity, muscle strength, cognition, respiratory health, and many other parameters can be measured automatically, periodically, and continuously. Automation has been developed to perform some measures, but not to a sufficient extent to substantially remove human labor from rearing and assays. Current smart cages are optimized for monitoring one mouse per cage instead of multiple mice. This is technically simple and acceptable for short-term studies of e.g. drug toxicity or metabolic activity. However, such smart cages are wholly incompatible with longevity studies, because for long-term housing, mice need to be co-housed with other mice in the same cage. This co-habitation is mandated by Association for Assessment and Accreditation of Laboratory Animal Care International (AAALAC) (the organization responsible for setting and validating good animal husbandry practices). Housing rodents singly for long periods of time (more than a week) is essentially animal torture, because it induces stress on the animal, substantially altering physiology, behavior, and decreasing lifespan. While cohabitation requirements could be handled by periodically moving mice between smart cages and their home cages throughout their lifespan, this is limited to female mice only, as removal of male mice from their home cage for more than 24 hours and subsequent replacement back into the home cage that contains other males causes fighting between males, again substantially altering their physiology and health.

Removing human labor from this process may allow a single lab or company to run an order of magnitude more experiments with the same budget and team and would remove many of the confounding factors caused by stress and operator differences in handling mice. Yet, while automated monitoring cages have existed for over three decades, cages that are suitable for longevity studies (or long-term studies of any kind) have yet to be built.

Automated rearing: concurrently, while aged wild-type mice and rats can be obtained from commercial vendors, they may be prohibitively expensive for some enterprises per mouse over the mouse's lifetime. This means that an average study employing one hundred aged mice requires expenses that may be beyond the reach of many academic labs and a substantial impediment to commercial enterprises. Yet, the raw materials cost (food, water, and bedding) of a mouse over its lifetime may be comparatively insignificant when compared to the manual labor of rearing and assaying. Most of the cost of the animals comes from human care and overhead costs due to low or no automation and process optimization. Therefore, by systematically developing technologies to improve automation and optimization, an opportunity is available to make aging assays more effective and efficient.

Current smart cages further lack critical assay technology to measure phenotypes relevant to aging. The sensors/assays included in the cage vary depending on the system purchased. Absent is technology on the market that include sensors designed to enable measurement of critical aspects of health in the home cage, such as musculoskeletal performance, cognition, vision, and many others which are assayed in most longevity studies.

Current smart cages have prohibitively low throughput. Due to the requirement of one animal per cage and bulky cage setups, current smart cages require an inhibitory large spatial footprint to run well-powered aging studies. To accommodate the large numbers of animals routinely required in aging studies, a single cage must have a compact footprint and be able to measure several animals at once.

Current smart cages are highly laborious to maintain. Because they were not designed for longevity studies, current smart cages are incompatible with automated cage washing or disposable cages, meaning they need to be manually cleaned between every animal, to remove the smell cues that can affect their behavior, a laborious process to do at scale. The current hardware simply does not provide the capacity required to conduct aging studies routinely in an automated fashion.

Further, aging studies economics can benefit from Multi-Object Tracking because multiple test subjects can exist in the same space. Algorithms lack the incentive to report time complexity versus favoring accuracy, the preferred being to have no tradeoff. Multi-object tracking (MOT) algorithms typically focus on reporting accuracy metrics rather than time complexity because accuracy is generally considered more important in evaluating the performance of tracking algorithms. However, in many real-world applications, the computational efficiency of the algorithms is also crucial. Papers disclosed in this specification discuss the trade-off between accuracy and efficiency in MOT algorithms.

There is a need, therefore, in the market for smart cages purpose-built for longevity studies that can further perform those longevity studies economically.

### SUMMARY OF THE INVENTION

Disclosed is a smart cage system and method for housing and assaying multiple vertebrate animals including at least one inner housing assembly and at least one outer housing assembly. The housing assemblies each having a top portion, a bottom portion, and at least one side portion, the side portions which may be at least partly transparent. The inner housing assemblies are designed to be at least partly disposed within and removable from the outer housing assemblies. The housing assemblies, in some embodiments may be further disposed within at least one rack assembly designed to hold the plurality of at least one housing assemblies wherein housing assemblies may be operationally contained at least one or more of vertically and horizontally from each other. The housing assemblies in this at least one rack assembly may be at least partially removed from the rack independently from other housing assemblies.

The smart cages include at least one controller designed to monitor and record data from at least one or more sensors from a group of: optical sensors, motion sensors, pressure sensors, weight sensors, temperature sensors, humidity sensors, proximity sensors, chemical sensors, volume sensors, level sensors, audio sensors, odor sensors, heartbeat sensors, brainwave sensors, body mass sensors, color sensors, rotary sensors, light sensors, oscillation sensors, balance sensors, reflex or reaction sensors, waterflow sensors, force meter sensors, load sensors, electrical sensors, and bite strength sensors, the sensors designed to monitor at least one or more of the environment, a device within one or more of the housing assemblies, and at least one vertebrate animal within the inner housing assembly, wherein each smart cage may have unique configurations of at least one or more sensors.

The at least one or more sensors are operationally synchronized at least one or more of before, in real time, and after sensing an action, wherein data captured by each at least one or more sensors can be synchronized by way of at least one time measuring device. A multi object tracking software system is operationally coupled to at least one optical sensor by the at least one controller, the multi object tracking software designed to track individuals of the multiple vertebrate animals by way of at least one or more from a group of: object detection, object reidentification, generating trajectories, and aggregating features, the multi object tracking software further designed to use at least one or more from a group of: appearance models, motion models, interaction models, exclusion models, and occlusion handling.

At least the outer housing assembly includes at least one or more from a group of ports, slots, shelves, pockets, hooks, fasteners, and sleeves each designed to retain at least one sensor. At least one or more sensors may be operationally coupled to the outer housing assembly wherein the inner housing assembly may be removed without removing the at least one or more sensors operationally coupled to the outer housing assembly.

In some embodiments of the smart cage system and method for housing and assaying multiple vertebrate animals, at least one physiological software system is designed to track, from the data gathered from the at least one or more sensors and the a multi object tracking software system, measures of each of the multiple vertebrate animals from at least one or more from a group of: lifespan, frailty index, muscle strength, run endurance, learning and memory, balance and coordination, body weight, food intake, total time spent in sleep and awake, temporal pattern of being asleep and awake, speed of nest building, visual acuity, hearing acuity, water intake, coat color/density, position tracking, distance traveled, movement speed, sleep time, cardiovascular health, cognition, balance and coordination, tremors, gait deficiencies, vision movement, and speed of nest building.

In some embodiments of the smart cage system and method for housing and assaying multiple vertebrate animals, at least the inner housing assembly includes a cage floor on which may be disposed at least one run wheel and a a tray designed to contain animal feed.

In some embodiments of the smart cage system and method for housing and assaying multiple vertebrate animals, the top portion of the outer housing assembly includes at least one or more of: a controller designed to be a local hub to measure and integrate data associated with smart cage assays conducted using the at least one or more sensors, the controller operationally coupled at least one or more of wired and wirelessly, and at least one or more of directly or by way of at least one other computer, to report data to a central data processing system. At least one overhead LED screen is designed to cover at least a portion of a horizontal dimension of the smart cage. The overhead LED screen is used to display at least a looming spot for vision assays. At least one or more of an infrared camera and a near infrared camera is used to record video substantially continuously from the cage, the video designed to be used at least for individual animal position tracking. At least one or more of an infrared LED and a near infrared LED is designed to illuminate an interior portion of the inner smart cage. The infrared cameras may, in some embodiments, contain an 840 nm high-pass filter adapted to prevent interference by visible light from the LED screen.

In some embodiments of the smart cage system and method for housing and assaying multiple vertebrate animals, the side portion of the outer housing assembly includes at least one or more of: a control panel which may be at least one or more of an LED touchscreen and a panel with buttons designed for mouse learning and memory assays without physical contact, at least one air valve, at least one main water dispenser and valve, at least one secondary water dispenser and valve for reward administration, at least one food dispenser and valve, at least one speaker and a microphone designed for hearing acuity testing, at least one force meter for a grip bar for muscle strength testing, at least one force meter for weight estimation, and at least one force meter for bite strength estimation.

In some embodiments of the smart cage system and method for housing and assaying multiple vertebrate animals, a robotic arm assembly is operationally coupled to move horizontally and vertically substantially along the entirety of the height and width of the rack assembly and is further designed to remove housing assemblies at least partly from the rack.

The smart cage and robotic arm system may contain an automated restraining, anesthetizing, injection and blood collection system to perform injections and blood draws on vertebrate animals within the smart cage system.

### BRIEF DESCRIPTION OF THE DRAWINGS

FIG. 1 illustrates a profile view of a representative smart cage system wherein interior components are viewable for illustration through sides that may be otherwise opaque.

FIG. 2A illustrates a profile view of an outer housing of the representative smart cage system.

FIG. 2B illustrates a profile view of the outer housing of the representative smart cage system wherein interior components are non-viewable through substantially solid sides.

FIG. 3 illustrates a representative inner housing of the representative smart cage system.

FIG. 4 illustrates a representative controller and sensor system.

FIG. 5 illustrates a representative multi-tracking software system.

FIG. 6 illustrates a representative object detection computer vision approach.

FIG. 7 illustrates a representative software-based approach to generating trajectories.

FIG. 8 illustrates representative mouse vectors within the representative smart cage.

FIG. 9 illustrates representative aggregating features of a motion object tracking program.

FIG. 10A illustrates a profile view of a representative rack assembly for the representative smart cage system.

FIG. 10B illustrates a front view of a representative rack assembly for the representative smart cage system.

FIG. 11 illustrates a cross-sectional section of the representative at least one rack assembly.

FIG. 12 illustrates a top view of the representative at least one rack assembly.

FIG. 13 illustrates a robotic arm assembly.

FIGS. 14A to 14C illustrate a smart cage for housing and assaying method for multiple vertebrate animals.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

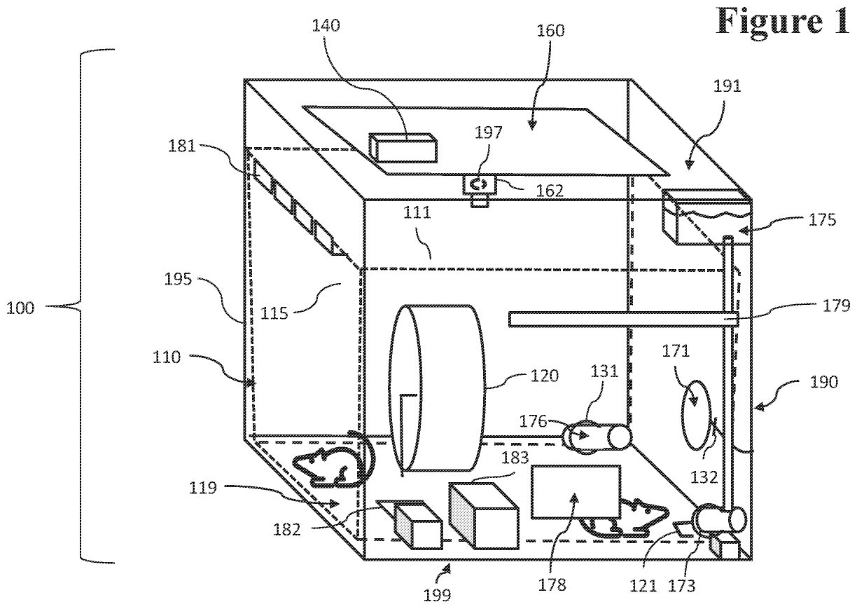

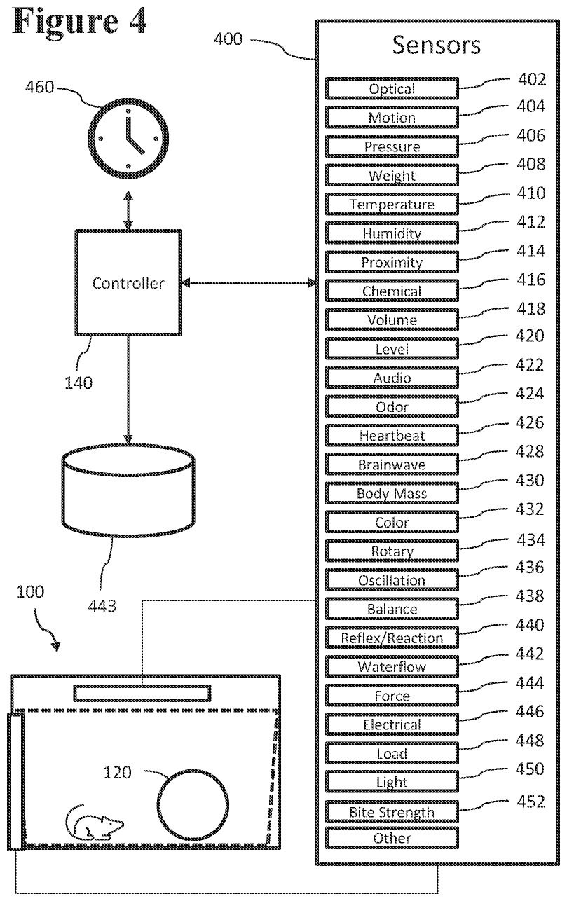

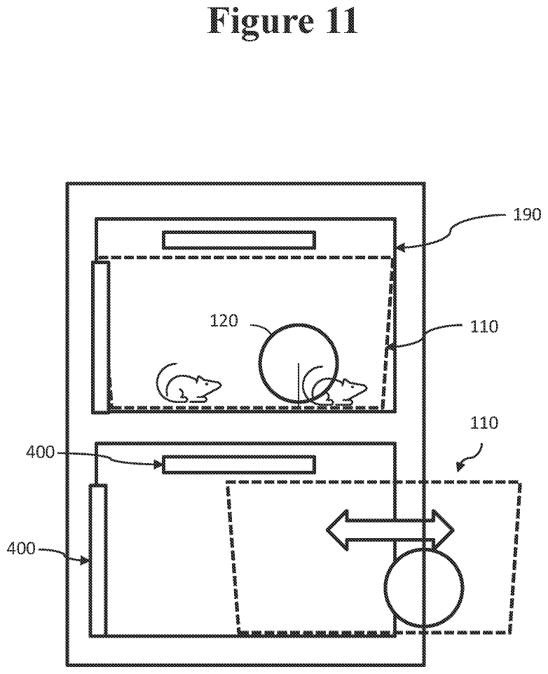

FIG. 1 illustrates a representative smart cage system 100 for housing and assaying multiple vertebrate animals including at least one inner housing assembly 110 and at least one outer housing assembly 190. The inner housing assembly has a top portion 111, a bottom portion 119, and at least one side portion 115, where, in the preferred embodiment, the at least one side portion 115 is at least partly transparent. The outer housing assembly has a top portion 191, a bottom portion 199, and at least one side portion 195. As illustrated in FIG. 11, the inner housing assemblies 110 are designed to be at least partly disposed within and removable from the outer housing assemblies 190 wherein certain elements such as sensors 400, listed in FIG. 4, may remain in place with the outer housing assemblies 190 when the inner housing assemblies 110 are removed. In this way, the inner housing assemblies 110 may be removed from the outer housing assemblies 190 for such tasks as cleaning or replacement without removing sensors 400 and other parts of the outer housing assemblies that need not be cleaned or replaced. Many configurations of sensors 400 may be designed as suitable for given assays, and multiple elements may be included such as multiple run wheels 120 though only one run wheel 120 is illustrated in this representative embodiment. The smart cage system 100 is designed for custom configurations, as well as for standardization such as to ensure smart cages 100 for a given assay can be configured to best fit that assay and then, for the given assay, be uniform across test animals for that assay. Vertebrate animals include, but are not limited to, mice and rats from the Family Muridae.

FIG. 1 further illustrates that in some embodiments of the smart cage system 100 for housing and assaying multiple vertebrate animals, at least the inner housing assembly 110 includes a bottom portion or cage floor 119 on which may be disposed at least one run wheel 120 and tray designed to contain animal feed. A given outer housing assembly 190 design is designed to be paired with a given compatible inner housing 110 design, for example, so that parts 131 align with given at least one sensor 400.

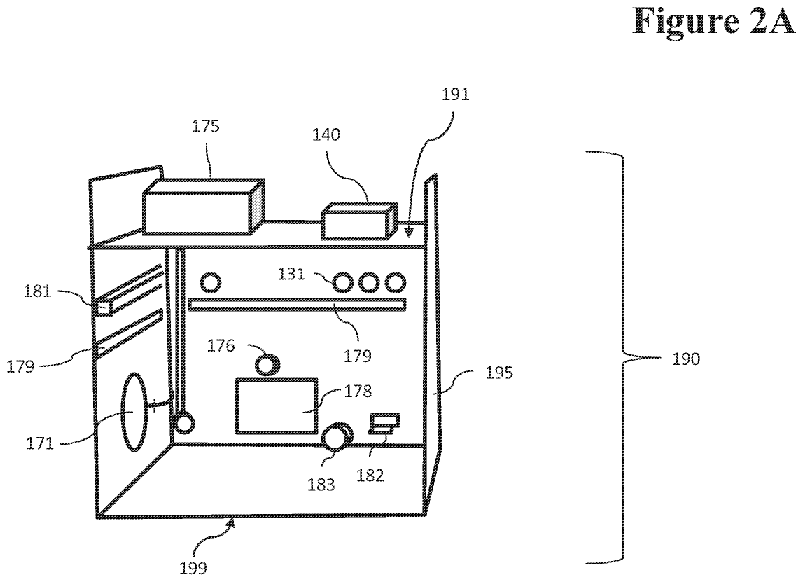

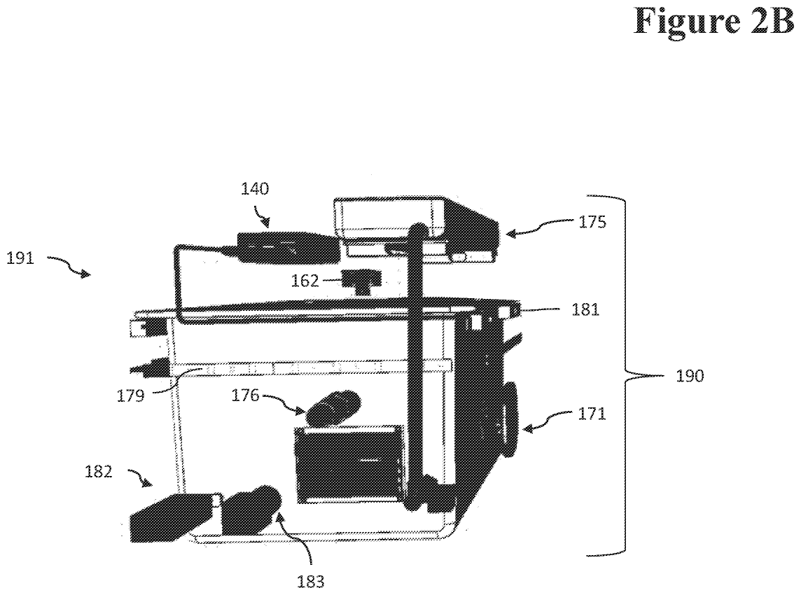

FIGS. 1, 2A, and 2B illustrate that the housing assemblies 110, 190 may include at least one or more from an attachment group of ports, slots, shelves, pockets, hooks, fasteners, and sleeves each designed to retain at least one sensor—a port 131 disposed in the inner and outer housing assemblies 110, 190 and a clip 132 disposed on the outer housing assembly 190 being illustrated in the representative embodiment. No specific arrangement from the attachment group is required except that it suits particular assays and, such as with the ports 131 of the inner housing assembly 110, facilitates removing the inner cage assembly 110 without necessarily removing the given sensors 400. As such, the disclosed smart cage system 100 may be custom manufactured for a particular assay and may be manufactured as multiple smart cages 100 with the same at least one or more ports, slots, shelves, pockets, hooks, fasteners, and sleeves as required for the given assays.

FIGS. 1, 2A, and 2B further illustrate that in some embodiments of the smart cage system 100 for housing and assaying multiple vertebrate animals, the top portion of the outer housing assembly 191 includes at least one or more of: a controller 140 designed to be a local hub to measure and integrate data associated with smart cage assays conducted using the at least one or more sensors 400, the controller 140, which may be termed a computer in the sense that the heart of a computer is its controller, a system of memory included, operationally coupled at least one or more of wired and wirelessly, and at least one or more of directly or by way of at least one other computer to report data to, and as further illustrated in FIG. 4, to a central data processing system 443. At least one overhead LED screen 160 is designed to cover at least a portion of a horizontal dimension of the smart cage 100 in the representative embodiment. The overhead LED screen 160 on the representative embodiment is used to display at least a looming spot for vision assays, for example, to observe defensive mice responses to overhead activity. At least one or more infrared cameras which may be an infrared wide-field camera and a near infrared wide-field camera 162, which in some embodiments may be operationally coupled to the center of the LED screen, is adapted to record video substantially continuously from the cage, the video designed to be used at least for individual animal position tracking. Other camera types may be used such as standard ROYGBIV light cameras designed to record light visible to people. At least one or more of an infrared LED and a near infrared LED 179 is designed to illuminate an interior portion of the inner smart cage and may be disposed on an at least one LED Strip. In some embodiments, at least one pass filter 197 may be disposed at least one or more of in front and behind the infrared camera lenses.

FIGS. 1, 2A, and 2B further illustrate that in some embodiments of the smart cage system 100 for housing and assaying multiple vertebrate animals, the side portion of the outer housing assembly 195 includes at least one or more of: a control panel 178 which may be at least one or more of an LED touchscreen and a panel with buttons designed for conducting learning and memory assays without physical human contact with the vertebrate animal, such as a mouse. Also include on the outer housing assembly 190 and designed to be operationally useful within the inner housing assembly 110 are at least one air valve 176, at least one main water dispenser and valve 173, and may include at least one secondary water dispenser and valve for reward administration, at least one food dispenser and valve, at least one speaker and a microphone designed 171 for hearing acuity testing, at least one force meter for a grip bar 182 for muscle strength testing, and at least one force meter 408 for weight estimation, and at least one force meter for bite strength estimation 552. One representative embodiment is a flat Flexiforce sensor adapted to measure the strength of mouse bite in the bite-weight-sensor. The inventive concept may also include such elements as a water dispensing system 175 that may be further designed to offer water as a reward. Such embodiments may include the main water dispenser and valve 173 and the secondary water dispenser for reward administration. Such embodiments may include a main food dispenser and valve and a secondary food dispenser for reward administration, a food dispenser otherwise being an expected element of a cage environment for life support of vertebrate animals. Other elements may be added necessarily for life support such as, on the representative embodiment, the air valve 176.

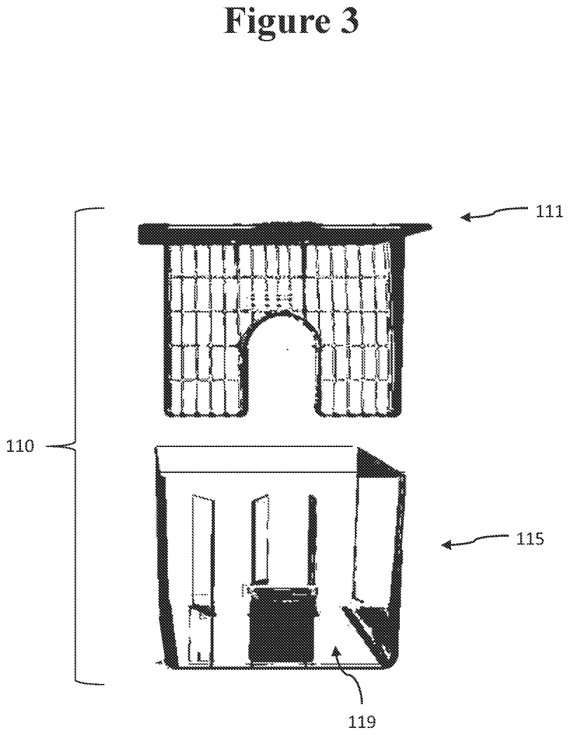

FIG. 3 illustrates a representative inner housing assembly 110 wherein the top portion if the inner housing assembly 111 is partly disposed within the bottom portion of the inner housing assembly 110. In other embodiments of the inner housing assembly 110, the top portion of the inner housing assembly 111 may be disposed substantially on the inn housing assembly side portion 115.

FIG. 4 illustrates that the smart cage assemblies 100 include the at least one controller 140 designed to monitor and record data from at least one or more sensors 400 from a group of: optical sensors 402, motion sensors 404, pressure sensors 406, weight sensors 408, temperature sensors 410, humidity sensors 412, proximity sensors 414, chemical sensors 416, volume sensors 418, level sensors 420, audio sensors 422, odor sensors 424, heartbeat sensors 426, brainwave sensors 428, body mass sensors 430, color sensors 432, rotary sensors 434, oscillation sensors 436, balance sensors 438, reflex or reaction sensors 440, waterflow sensors 442, force meter sensors 444, electrical sensors 446, load sensors 448, light sensors 550, and bite strength sensors 552, the sensors 400 designed to monitor at least one or more of the environment, a device within one or more of the housing assemblies, and at least one vertebrate animal within the inner housing assembly 110, wherein each smart cage 100 may have unique configurations of at least one or more sensors 400. Illustrated in this representative embodiment are load sensors 181, a grip bar form of a force meter sensor 182, and a balance beam form of a balance sensor 183. Many types of sensors 400 may be employed, depending upon the assays needed, including sensors 400 that may be unlisted in this disclosure.

FIG. 4 further illustrates that the at least one or more sensors 400 are operationally synchronized at least one or more of before, in real time, and after sensing an action, wherein data captured by each at least one or more sensors 400 can be synchronized by way of at least one time measuring device 460 such as a clock or timer.

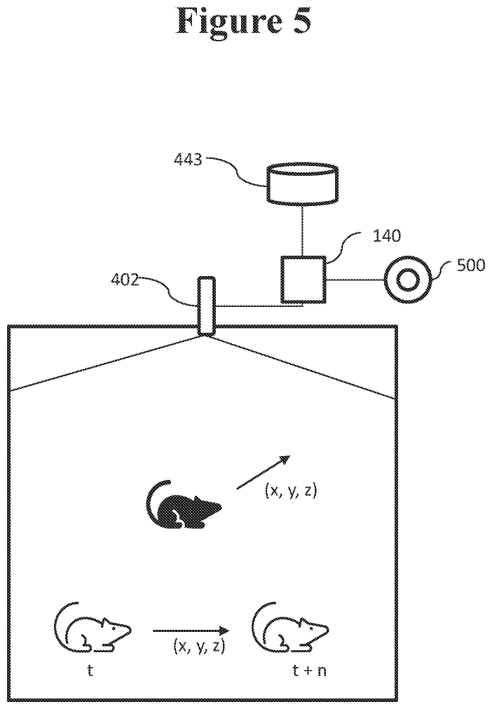

FIG. 5 illustrates that a multi object tracking software system is operationally coupled to at least one optical sensor 402 by the at least one controller 140. Multi object tracking software 500 is software capable of tracking and maintaining the identities of all individuals across most of the frames in a video. The Multi object tracking software 500 is designed to track individuals of the multiple vertebrate animals by way of at least one or more from a group of: object detection such, and as illustrated, as at time (t), object reidentification such as at time (t+n), where (n) represents a given interval of time, generating trajectories such as vector (x, y, z), the vector having a direction and a magnitude, the vector which may be the summation of multiple other vectors, and subject features such as vertebrate animal color, shape, size, marks, tags including imbedded tags, other identifying features, and aggregated features.

In some embodiments, the vertebrate animals may have tattoos such as on the tails or ears of mice, the tattoos having patterns such as 1-4 black stripes, dots, or letters, that can be identified by computer vision. Therefore, animals may be tracked and location data synced with sensor data to identify which individual animals produced what sensor data.

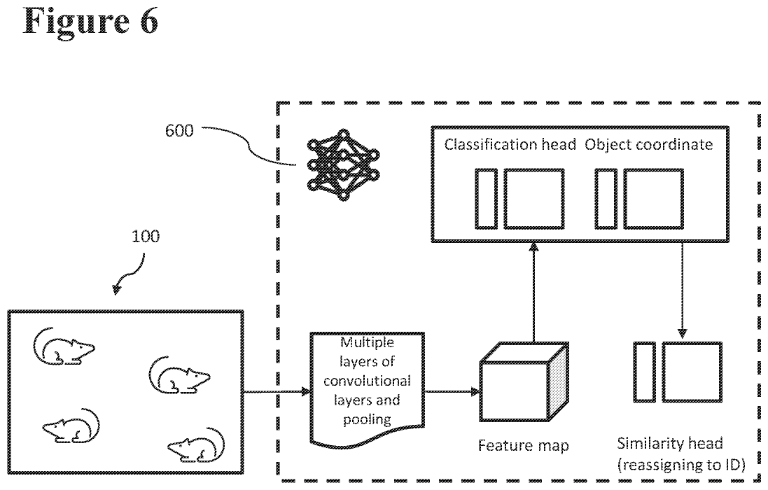

FIG. 6 illustrates representative object detection as a computer vision approach operationally coupled to a neural network software program 600 designed to detect individuals displayed on an image. Multiple layers of convolution and pooling create a tensor feature map of sufficient detail that is then used in the final classification layer and object coordinates heads. A tensor is defined as an algebraic object that describes a multilinear relationship between sets of algebraic objects related to a vector space and that may map between different objects such as vectors, scalars, and other tensors. The layers and their corresponding weights are updated with the backpropagation algorithm.

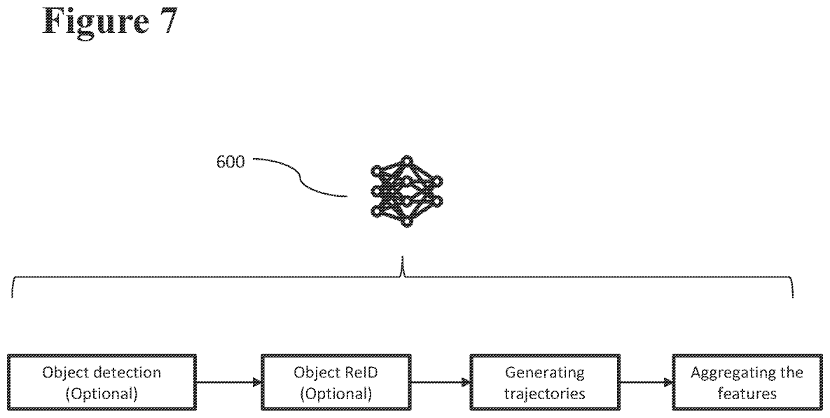

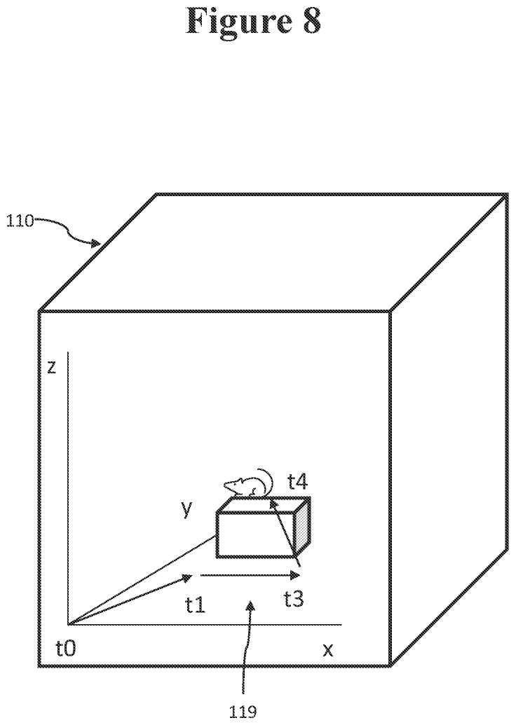

FIG. 7 illustrates object reidentification, sometimes abbreviated as ReID, which is a computer vision approach wherein a representative neural network software program 600, or other software system designed for multi object tracking, assigns and, as required reconfirms distinct identities of all the individuals on an image. Included are object detection, object reidentification, generating trajectory, and aggregating the features as related to FIG. 8.

FIG. 8 illustrates that generating trajectories is a software-based approach wherein visual cues created from object detection and object ReID differentials across at least a portion of frames, frames being images captures such as by video, to produce trajectories of the objects throughout at least the relative portion of a video to include at least one or more of a probabilistic approach wherein algorithms such as Kaman filters and Particle filters are designed to track objects. Kalman filter and the Particle filter approaches are based on probabilistic inferences represent states of objects as a distribution with uncertainty. The tracking algorithm, for example, might be a series of vectors (x, y, z,) over a period of time (t) with intervals n such as [xt+yt+zt]+[x(t+1), y(t+1), z(t+1)] . . . +[x(t+n), +y(t+n), +z(t+n)] wherein the vectors can be used to determine where a given vertebrate animal was at a given time and such other values as direction of travel, speed of travel, total distance traveled, and certain predictive values such as where the vertebrate animal will likely be next. Illustrated in FIG. 8 is one simplified vector track wherein a mouse moves from a space on the cage floor 119 of the representative inner housing assembly 110 to a space atop a shelf within the inner housing assembly 110, illustrating the multi object tracking software 500 can measure three-dimensional as well as two-dimensional movement across time.

The goal of the tracking algorithms is to estimate the probabilistic distribution of target states by a variety of probability reasoning methods based on existing observations. This kind of approach typically requires only existing past and present observations and may include any number of derivatives between a given start and end point of a subject such as a mouse, and are, therefore, appropriate for the task of online tracking. As only the existing observations are employed for estimation, assumptions of Markov properties may be made in the object's state sequence-meaning substantially the computer system 140 may guess where the object was located between given intervals, such as t1.5 between t1 and t2 where the locations t1 and t2 was known and where, for illustration, t1 and t2 might be an interval such as 1/30^{th} of a second as would be a typical video frame rate or may be a longer interval such as every half second or every second as to balance the granularity of tracking data needed with the data storage required.

Deterministic approaches apply algorithms such as Hungarian matching, Bipartite graph matching, Dynamic programming, Min-cost max-flow network flow, Conditional random field, and the maximum-weight independent set (MWIS). As opposed to the probabilistic inference methods, approaches based on deterministic optimization aim to find the maximum a posteriori (MAP) solution to multi object tracking that obtains a point estimate of unobserved quantity based on object data prior distributions-substantially an optimization problem.

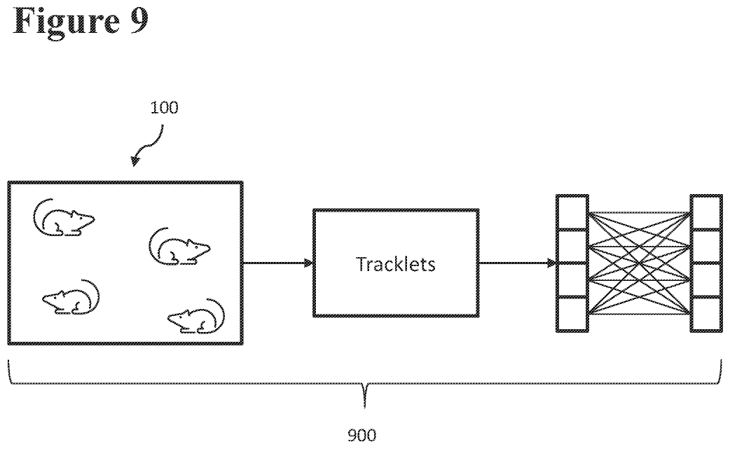

FIG. 9 illustrates that aggregating features of the motion object tracking program take data from the aggregate of tracking information and reconcile measurements of one or more sensors 400 such as bite strength and hearing acuity, designed, therefore, to assign sensor data to its associated distinct object such as a particular mouse wherein tacking data from one tracking algorithm is designed to be cross-checked by one or more other tracking algorithms.

The multi object tracking software is further designed to use at least one or more from a group of: appearance models, motion models, interaction models, exclusion models, and occlusion handling.

An appearance model includes two core components: 1) visual representation and 2) statistical measuring. Visual representation describes the visual characteristics of an object using some features, either based on a single cue or multiple cues. Statistical measuring, on the other hand, is the computation of similarity between different observations.

Motion models capture the dynamic behavior of an object. It estimates the potential position of objects in future frames, thereby, reducing the search space. In most cases, objects are assumed to move smoothly in the world and, therefore, in the image space (except for abrupt motions). Two kinds of motion models are implemented, 1) linear motion models and 2) non-linear motion models. A linear motion model is commonly used to explain the object's dynamics. However, there are some cases linear motion models are not well-suited to handle. To this end, non-linear motion models 900 are proposed to produce, as noted in FIG. 9, more accurate motion affinity between tracklets, tracklets being defined as a fragment of the track followed by a moving object, as constructed by an image recognition system such as the multi object tracking system. Nonlinear dynamical systems describe changes in variables over time and, as might seem to an observer of one or more mice, may appear chaotic, unpredictable, or counterintuitive, contrasting with linear systems.

Interaction models—The interaction model, also known as mutual motion models, captures the influence one object may have on other objects. In the cage scenery, an object would experience some “force” from other mice, or other vertebrate animal test subjects, and objects. For instance, when mice are moving about a cage, the mice would adjust speed, direction, and destination to avoid collisions with other mice. Another example is when a crowd of mice seek their way through a door. Each mouse follows some mice and leads others at the same time. Two typical interaction models in representative embodiments of the disclosed inventive concept include social force models and crowd motion pattern models.

Exclusion models include, in representative embodiments of the inventive concept, constraints to avoid physical collisions when seeking solutions to multi object transfer models. Exclusion models account for the fact that two distinct objects cannot occupy the same physical space in the real world and so should not do so within a virtual model of the real world. Given multiple detection responses and multiple trajectory hypotheses, generally there are two constraints. The first constraint is detection level exclusion, meaning that two different detected objects in the same frame cannot be assigned as being the same entity or, as may be termed, the same target. The second constraint is trajectory-level exclusion, i.e., two trajectories cannot be infinitely close to each other. In short, the object cannot be in the same space nor, if in the same space, having the same vector, though vertebrate animals being living organisms, tracked subjects such as mice may compete to occupy a space, and resulting tussles between mice can further necessitate superior multi object tracking. As a data-saving element, the interval (t) tracked may be varied wherein the intervals grow shorter when the number of vertebrate animals or their activity increase generally or in a specific location of the smart cage.

Occlusion handling models are designed to mitigate identification switches and trajectory fragmentation and include 1) at least one or more of tracking only a visible portion of on object partly obscured by another object while inferring the state of the whole object, 2) a hypothesizing and testing strategy according to observations of object appearances and trajectories from previous frames, 3) buffer and recover wherein the states of objects a recovered from frame before occlusion took place, and 4) unique markers wherein an object can be seen as unique because of differences in at least one or more of size, shape, color, and tag wherein the tag may further be visually or electronically detectable.

Examples of some of the individual features that are end results from the elements previously presented as could, therefore, be assigned to one object such as a mouse, the tracking results reconciled with sensor data, at least one or more from a group of: movement speed, rearing events and time, sleep time, feeding events, and strength.

In some embodiments of the smart cage system 100 for housing and assaying multiple vertebrate animals, at least one physiological software system is designed to track, from the data gathered from the at least one or more sensors 400 and the multi object tracking software system, measures of each of the multiple vertebrate animals from at least one or more from a group of: lifespan, frailty index, muscle strength, run endurance, learning and memory, balance and coordination, body weight, food intake, total time spent in sleep and awake, temporal pattern of being asleep and awake, speed of nest building, visual acuity, hearing acuity, water intake, coat color/density, position tracking, distance traveled, movement speed, sleep time, cardiovascular health, cognition, balance and coordination, tremors, gait deficiencies, vision movement, and speed of nest building.

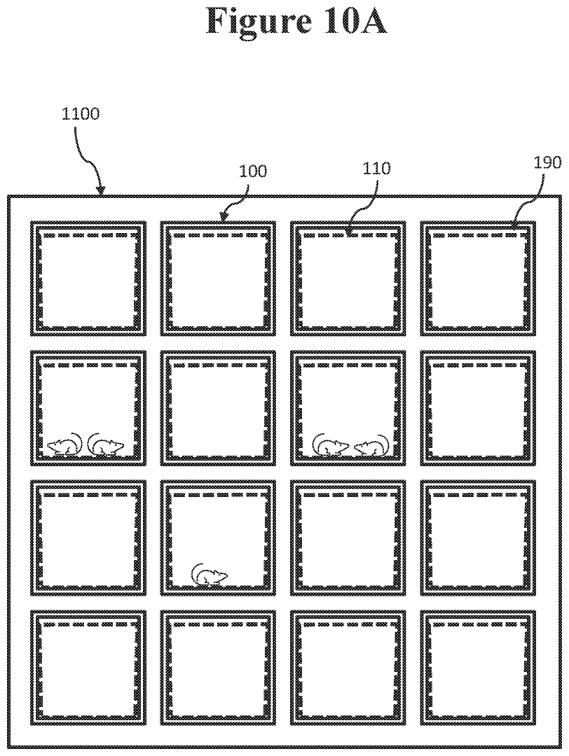

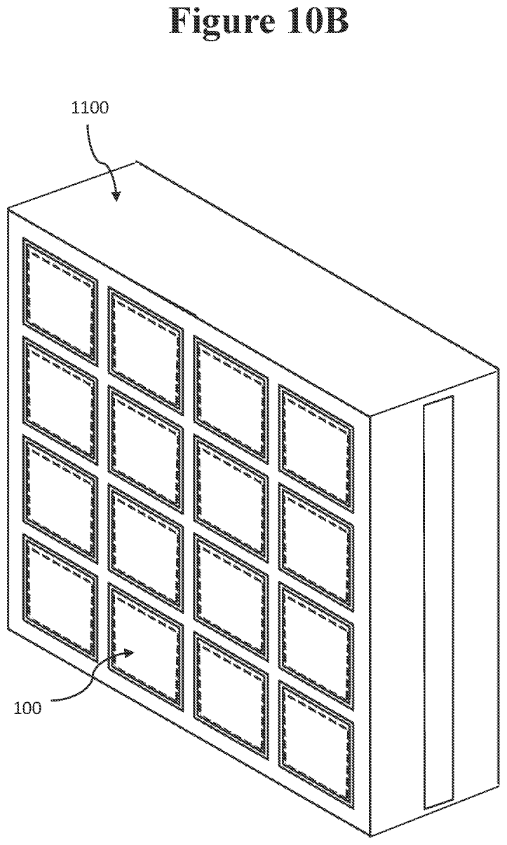

FIGS. 10A and 10B illustrate that the smart cages 100, in some embodiments, may be further disposed within at least one rack assembly 1100 designed to hold a plurality of at least one housing assemblies 110, 190 wherein smart cages 100 may be operationally contained at least one or more of vertically and horizontally from each other. Rack assemblies 1100, like bookshelves in a library, may be configured in many ways to fit the space available in a room, and other features may be included such as ladders and lifts wherein, as needed, users can access given smart cages 100 that may be inaccessible from the room floor. Further elements of the rack assembly 1100 may allow smart cages to be operationally connected as by a pathway wherein vertebrate animals such as mice may move from one inner housing assembly 110 to another inner housing assembly 110.

FIG. 11 illustrates a cross-sectional section of the representative at least one rack assembly 1100 wherein one inner housing assembly 110 is illustrated disposed within one outer housing assembly 190 and another inner housing assembly 110 is illustrated partly removed from another outer housing assembly 190. Smart cages 100 may be at least partially removed from the rack assemblies 1100 independently from other smart cages 100.

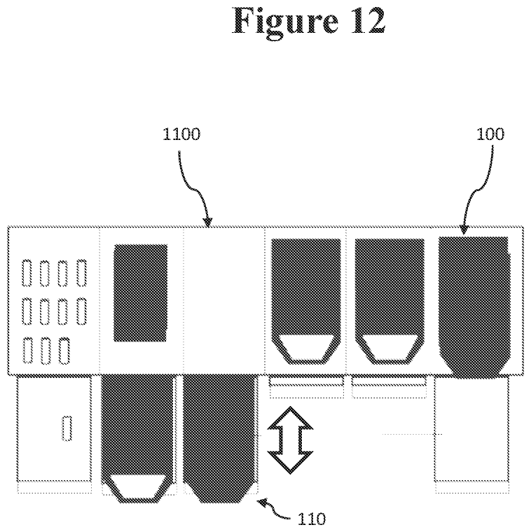

FIG. 12 illustrates a top view of the representative at least one rack assembly 1100 wherein some housing assemblies 110, 190 are partly removed from the representative rack assembly 1100.

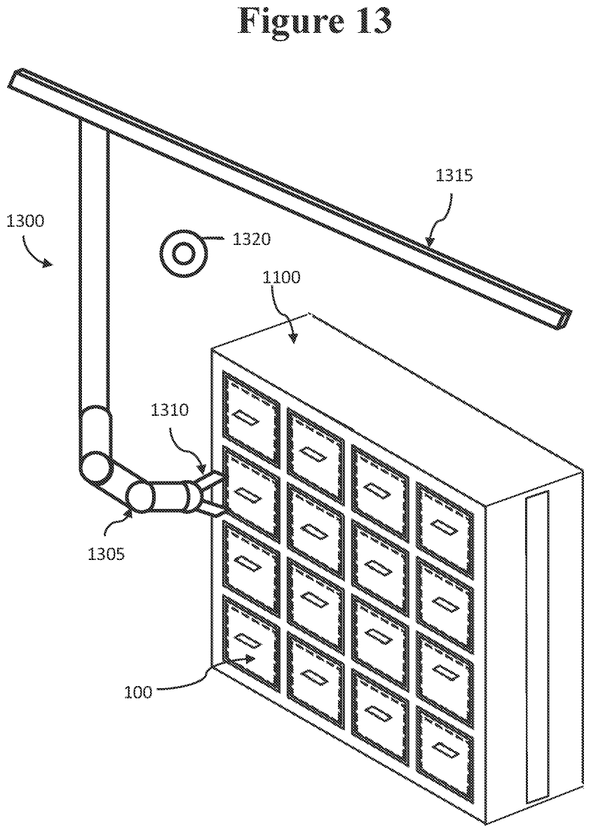

FIG. 13 illustrates that in some embodiments of the smart cage system 100 for housing and assaying multiple vertebrate animals, a robotic arm assembly 1300 is operationally coupled to move horizontally and vertically substantially along the entirety of the height and width of the rack assembly 1100 and is further designed to remove housing assemblies at least partly from the rack. The track system and articulation joints 1305 of the robot arm allow grippers 1310 to be positioned as required and moved along vectors of a three-dimensional space that permits at least partial removal of smart cage systems 100 from the associated rack assembly 1100. Robot arm systems 1300 may be apart from the rack assembly 1100 such as those that may be disposed on an autonomous ground vehicle or on an independent rail system.

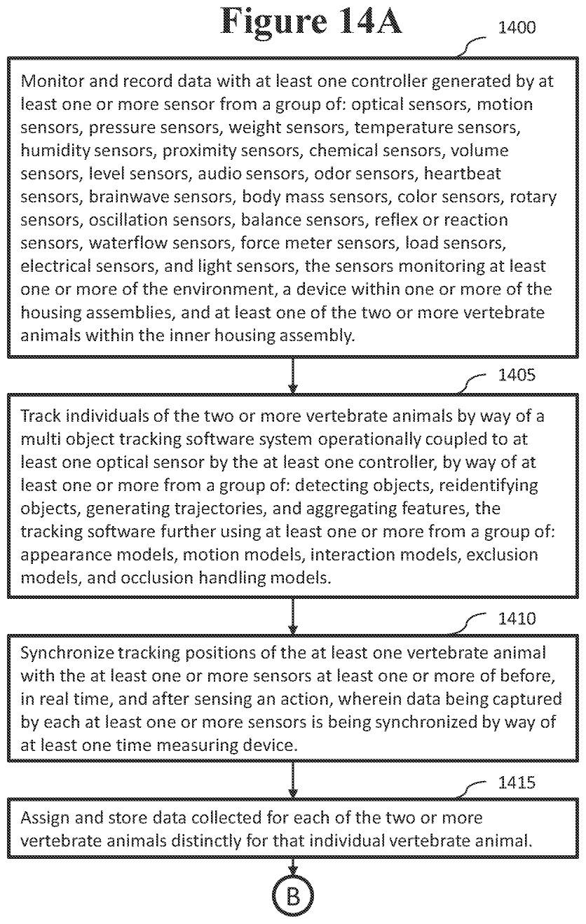

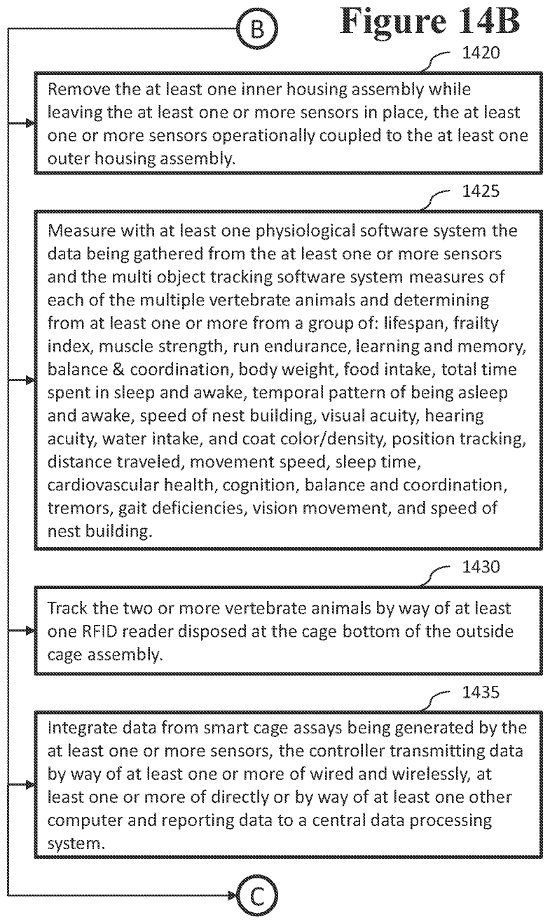

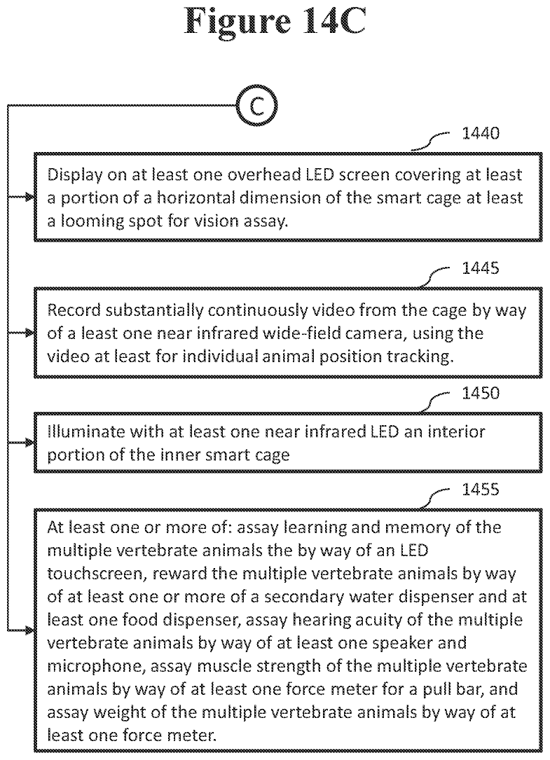

FIGS. 14A to 14C illustrate the smart cage for housing and assaying method for multiple vertebrate animals including housing, feeding, and hydrating two or more vertebrate animals within at least one inner housing assembly 110. The method includes the step of 1400, monitoring and recording data with the at least one controller generated by the at least one or more sensors 400 from the group of: optical sensors, motion sensors, pressure sensors, weight sensors, temperature sensors, humidity sensors, proximity sensors, chemical sensors, volume sensors, level sensors, audio sensors, odor sensors, heartbeat sensors, brainwave sensors, body mass sensors, color sensors, rotary sensors, oscillation sensors, balance sensors, reflex or reaction sensors, waterflow sensors, force meter sensors, load sensors, electrical sensors, and light sensors, the sensors 400 monitoring the at least one or more of the environment, devices within the one or more of the housing assemblies, and the at least one of the two or more vertebrate animals within the inner housing assembly 110. The method includes the step of 1405, synchronizing the at least one or more sensors 400 at least one or more of before, in real time, and after sensing the action, wherein data being captured by each of the at least one or more sensors 400 is being synchronized by way of the at least one time measuring device 460. The method includes the step of 1410, tracking individuals of the two or more vertebrate animals by way of the multi object tracking software system operationally coupled to the at least one optical sensor 402 by the at least one controller 140, by way of the at least one or more from the group of: detecting objects, reidentifying objects, generating trajectories, and aggregating features, the multi object tracking software further using the at least one or more from the group of: appearance models, motion models, interaction models, exclusion models, and occlusion handling models, the tracking of individual vertebrate animals adapted to be time synchronized. The method includes the step of 1415, assigning and storing data collected for each of the two or more vertebrate animals distinctly for that individual vertebrate animal.

The method may include the step of 1420, including removing the at least one inner housing assembly 110 while leaving the at least one or more sensors 400 in place, the at least one or more sensors 400 operationally coupled to the at least one outer housing assembly 190.

The method may include the step of 1425, including measuring with at least one physiological software system the data being gathered from the at least one or more sensors 400 and the multi object tracking software system measures of each of the multiple vertebrate animals and determining from the at least one or more from the group of: lifespan, frailty index, muscle strength, run endurance, learning and memory, balance and coordination, body weight, food intake, total time spent in sleep and awake, temporal pattern of being asleep and awake, speed of nest building, visual acuity, hearing acuity, water intake, and coat color/density, position tracking, distance traveled, movement speed, sleep time, cardiovascular health, cognition, balance and coordination, tremors, gait deficiencies, vision movement, and speed of nest building.

The method may include the step of 1430, including tracking the two or more vertebrate animals by way of the at least one RFID reader disposed at the cage bottom of the outside cage assembly 199, though other locations of the RFID reader may be used The method may include the step of 1435, including integrating data from smart cage assays being generated by the at least one or more sensors 400, the controller 140 transmitting data by way of at least one or more of wired and wirelessly, at least one or more of directly or by way of at least one other computer and reporting data to the central data processing system 443. The method may include the step of 1440, displaying on the at least one overhead LED screen 160 covering at least the portion of the horizontal dimension of the smart cage at least the looming spot for vision assays. The method may include the step of 1445, substantially continuously recording video from the cage by way of the least one near infrared camera 162 that, in some embodiments, may be attached to the center of the LED screen 160, using the video at least for individual animal position tracking. The method may include the step of 1450, illuminating with At least one or more of an infrared LED and a near infrared LED the interior portion of the inner smart cage 110.

The method may include the step of 1455, including at least one or more of: assaying learning and memory of the multiple vertebrate animals the by way of the control panel 178, rewarding the multiple vertebrate animals by way of the at least one or more of the secondary water dispenser and the at least one food dispenser, assaying hearing acuity of the multiple vertebrate animals by way of the at least one speaker and microphone 171, assaying muscle strength of the multiple vertebrate animals by way of the at least one force meter for the grip bar 182, and assaying weight of the multiple vertebrate animals by way of the at least one weight sensor 408, which may be termed a scale.

The following patents are incorporated by reference in their entireties: U.S. Pat. Nos. 11,330,804, 11,109,801, 10,905,094, 10,420,503, 10,278,361, 9,516,857, 8,739,737, 8,161,910, 10,575,495, 10,973,202, 11,129,358, 5,000,120, 6,308,660, 5,307,757, 4,699,088, 5,513,596, 5,148,766, 5,894,816, 6,357,393, 10,634,548, 10,064,392, 10,709,110, 9,986,716, 10,398,316, US20230046736, US20190183097, US20180007862, US20180103609, US20170000081, US2019018309, US20190037800, US20190167178, US20170105385, US20170108369, US20190183089, US20180092605, CN 217694864, CN114586689, CN216627035, CN110583501, CN108812363, CN107278929, and EP2034815.

The following literature references are incorporated by reference in their entireties:
Singh S, Bermudez-Contreras E, Nazari M, Sutherland R J, Mohajerani M H Low-cost solution for rodent home-cage behaviour monitoring. PLOS ONE 14 (8): e0220751 (2019).
Chen, Z. et al. Automated, high-dimensional evaluation of physiological aging and resilience in outbred mice. Elife 11, (2022).
Kalliokoski, O. et al. Mice Do Not Habituate to Metabolism Cage Housing—A Three Week Study of Male BALB/c Mice. PLOS One 8, e58460 (2013).
Bellantuono, I. et al. A toolbox for the longitudinal assessment of healthspan in aging mice. Nat Protoc 15, 540-574 (2020).
Brown, S. D. M. et al. High-throughput mouse phenomics for characterizing mammalian gene function. Nat. Rev. Genet. 19, 357-370 (2018).
Whitehead, J. C. et al. A clinical frailty index in aging mice: Comparisons with frailty index data in humans. Journals Gerontol.—Ser. A Biol. Sci. Med. Sci. 69, 621-632 (2014).
Ackert-Bicknell, C. L. et al. Aging Research Using Mouse Models. Curr. Protoc. Mouse Biol. 5, 95-133 (2015).
Justice, J. N. et al. Battery of behavioral tests in mice that models age-associated changes in human motor function. AGE 2013 362 36, 583-595 (2013).
Deacon, R. M. J. Measuring the Strength of Mice. JoVE (Journal Vis. Exp. e2610 (2013) doi: 10.3791/2610.
Smith, J. P., Hicks, P. S., Ortiz, L. R., Martinez, M. J. & Mandler, R. N. Quantitative measurement of muscle strength in the mouse. J. Neurosci. Methods 62, 15-19 (1995).
Wimmer, M. E., Hernandez, P. J., Blackwell, J. & Abel, T. Aging impairs hippocampus-dependent long-term memory for object location in mice. Neurobiol. Aging 33, 2220-2224 (2012).
Wimmer, M. E. et al. Aging in Mice Reduces the Ability to Sustain Sleep/Wake States. PLOS One 8, e81880 (2013).
Deacon, R. M. J. Assessing nest building in mice. Nat. Protoc. 2006 13 1, 1117-1119 (2006).
Storchi, R. et al. Measuring vision using innate behaviours in mice with intact and impaired retina function. Sci. Reports 2019 91 9, 1-16 (2019).
Jussi Jero, D. E. C. A. K. L. The Use of Preyer's Reflex in Evaluation of Hearing in Mice. http://dx.doi.org/10.1080/00016480118142 121, 585-589 (2009).
Ounpraseuth, S. et al. A Method to Quantify Mouse Coat-Color Proportions. PLOS One 4, e5414 (2009).
Bewley, A., Ge, Z., Ott, L., Ramos, F., & Upcroft, B. (2016). Simple online and realtime tracking. In 2016 IEEE International Conference on Image Processing (ICIP) (pp. 3464-3468). IEEE.
Wojke, N., Bewley, A., & Paulus, D. (2017). Simple online and realtime tracking with a deep association metric. In 2017 IEEE International Conference on Image Processing (ICIP) (pp. 3645-3649). IEEE.
Bergmann, P., Meinhardt, T., & Leal-Taixé, L. (2019). Tracking without bells and whistles. In Proceedings of the IEEE/CVF International Conference on Computer Vision (pp. 941-951).
Milan, A., Schindler, K., & Roth, S. (2016). Challenges of ground truth evaluation of multi-target tracking. In 2016 IEEE Conference on Computer Vision and Pattern Recognition Workshops (CVPRW) (pp. 735-742). IEEE.
Leal-Taixé, L., Milan, A., Reid, I., Roth, S., & Schindler, K. (2017). Tracking the trackers: An analysis of the state of the art in multiple object tracking. arXiv preprint arXiv: 1705.02750.
Bewley, A., Ge, Z., Ott, L., Ramos, F., & Upcroft, B. (2016). Simple online and realtime tracking. In 2016 IEEE International Conference on Image Processing (ICIP) (pp. 3464-3468). IEEE.
Luo, W., Xing, J., & Milan, A. (2014). Multiple object tracking: A literature review. arXiv preprint arXiv: 1409.7618.
Kim, C., Li, F., Ciptadi, A., & Rehg, J. M. (2015). Multiple hypothesis tracking revisited. In Proceedings of the IEEE International Conference on Computer Vision (pp. 4696-4704).
Voigtlaender, P., Krause, M., Osep, A., Luiten, J., Sekar, B. B. G., Geiger, A., & Leibe, B. (2019). Mots: Multi-object tracking and segmentation. In Proceedings of the IEEE/CVF Conference on Computer Vision and Pattern Recognition (pp. 7942-7951).
Bergmann, P., Meinhardt, T., & Leal-Taixé, L. (2019). Tracking without bells and whistles. In Proceedings of the IEEE/CVF International Conference on Computer Vision (pp. 941-951).
Bochinski, E., Senst, T., & Sikora, T. (2020). Understanding the limitations of CNN-based multi-object tracking. In Proceedings of the 28th ACM International Conference on Multimedia (pp. 3152-3160).
Wang, Q., Zhou, L., Zhang, Z., & Qi, H. (2020). Deep online learning for multi-object tracking and segmentation. In Proceedings of the IEEE/CVF Conference on Computer Vision and Pattern Recognition Workshops (pp. 886-887).
Zhang, H., & Patel, V. M. (2020). Joint multi-object tracking and segmentation with detection-refinement. In Proceedings of the European Conference on Computer Vision (ECCV) Workshops (pp. 332-345).

While inventive concepts have been described above in terms of specific embodiments, it is to be understood that the inventive concepts are not limited to these disclosed embodiments. Upon reading the teachings of this disclosure, many modifications and other embodiments of the inventive concepts will come to mind of those skilled in the art to which these inventive concepts pertain, and which are intended to be and are covered by both this disclosure and the appended claims. It is indeed intended that the scope of the inventive concepts should be determined by proper interpretation and construction of the appended claims and their legal equivalents, as understood by those of skill in the art relying upon the disclosure in this specification and the attached drawings.

1. A smart cage system for housing and assaying multiple vertebrate animals comprising:
at least one inner housing assembly and at least one outer housing assembly, the housing assemblies each having a top portion, a bottom portion, and at least one side portion, the inner housing assemblies adapted to be at least partly disposed within and removable from the outer housing assemblies;
the smart cage including at least one controller adapted to monitor and record data from at least one or more sensors from a group of: optical sensors, motion sensors, pressure sensors, weight sensors, temperature sensors, humidity sensors, proximity sensors, chemical sensors, volume sensors, level sensors, audio sensors, odor sensors, heartbeat sensors, brainwave sensors, bite force sensors, body mass sensors, color sensors, rotary sensors, light sensors, oscillation sensors, balance sensors, reflex or reaction sensors, waterflow sensors, force meter sensors, load sensors, electrical sensors, and bite strength sensors, the sensors adapted to monitor at least one or more of the environment, a device within one or more of the housing assemblies, and at least one vertebrate animal within the inner housing assembly;
the at least one or more sensors operationally synchronized at least one or more of before, in real time, and after sensing an action, wherein data captured by each at least one or more sensors can be synchronized by way of at least one time measuring device;
a multi object tracking software system operationally coupled to at least one optical sensor by the at least one controller, the multi object tracking software adapted to track individuals of the multiple vertebrate animals by way of at least one or more from a group of: object detection, object reidentification, generating trajectories, and aggregating features, the multi object tracking software further adapted to use at least one or more from a group of: appearance models, motion models, interaction models, exclusion models, and occlusion handling models, the tracking of individual vertebrate animals adapted to be time synchronized; and
at least the outer housing assembly including at least one or more from a group of ports, slots, shelves, pockets, hooks, fasteners, and sleeves each adapted to retain at least one sensor.

2. The smart cage system for housing and assaying multiple vertebrate animals of claim 1, wherein the at least one or more sensors is operationally coupled to the outer housing assembly wherein the inner housing assembly may be removed without removing the at least one or more sensors.

3. The smart cage system for housing and assaying multiple vertebrate animals of claim 1, wherein at least one physiological software system is adapted, from the data gathered from the at least one or more sensors and a multi object tracking software system measures of each of the multiple vertebrate animals from at least one or more from a group of: lifespan, frailty index, muscle strength, run endurance, learning and memory, balance and coordination, body weight, food intake, total time spent in sleep and awake, temporal pattern of being asleep and awake, speed of nest building, visual acuity, hearing acuity, water intake, and coat color/density, position tracking, distance traveled, movement speed, sleep time, cardiovascular health, cognition, balance and coordination, tremors, gait deficiencies, vision movement, and speed of nest building.

4. The smart cage system for housing and assaying multiple vertebrate animals of claim 1, wherein the inner housing assembly includes a cage floor on which is disposed at least one run wheel, and a tray disposed within the inner housing assembly to contain animal feed.

5. The smart cage system for housing and assaying multiple vertebrate animals of claim 1, wherein the top portion of the outer housing assembly includes at least one or more of: a controller adapted to be a local hub to measure and integrate data associated with smart cage assays conducted using the at least one or more sensors, the controller operationally coupled at least one or more of wired and wirelessly, at least one or more of directly or by way of at least one other computer to report data to a central data processing system;
at least one overhead LED screen adapted to cover at least a portion of a horizontal dimension of the smart cage, the overhead LED screen used to display at least a looming spot for vision assays;
at least one or more of an infrared camera and a near infrared camera adapted to record video substantially continuously from the cage, the video adapted to be used at least for individual animal position tracking; and
at least one or more of an infrared and a near infrared LED adapted to illuminate an interior portion of the inner smart cage.

6. The smart cage system for housing and assaying multiple vertebrate animals of claim 5, wherein at least one pass filter is disposed at least one or more of in front and behind at least one camera lens.

7. The smart cage system for housing and assaying multiple vertebrate animals of claim 1, wherein the side portion of the outer housing assembly includes at least one or more of: a control panel adapted for mouse learning and memory assays, at least one air valve, at least one main water dispenser and valve, at least one secondary water dispenser and valve adapted for reward administration, at least one speaker and a microphone adapted for hearing acuity testing, at least one force meter for a grip bar adapted for muscle strength testing, at least one force meter for weight estimation, and at least one force meter for bite strength estimation.

8. The smart cage system for housing and assaying multiple vertebrate animals of claim 1, wherein the outside cage assembly includes at least one RFID reader.

9. The smart cage system for housing and assaying multiple vertebrate animals of claim 1, wherein the smart cage may be operationally coupled to at least one second cage wherein the at least one second cage may be at least one or more of a second smart cage that is substantially similar to the first smart cage, a second smart cage that is different than the first smart cage, and a non-smart cage, wherein the animal may be permitted at least at some times to move to and from the smart cage to and from the other cages.

10. A smart cage for housing and assaying method for multiple vertebrate animals comprising:
housing, feeding, and hydrating two or more vertebrate animals within at least one inner housing assembly, the inner housing assembly disposed at least partly within at least one outer housing assembly, the housing assemblies each having a top portion, a bottom portion, and at least one side portion;
monitoring and recording data with at least one controller generated by at least one or more sensor from a group of: optical sensors, motion sensors, pressure sensors, weight sensors, temperature sensors, humidity sensors, proximity sensors, chemical sensors, volume sensors, level sensors, audio sensors, odor sensors, heartbeat sensors, brainwave sensors, body mass sensors, color sensors, rotary sensors, light sensors, oscillation sensors, balance sensors, reflex or reaction sensors, waterflow sensors, force meter sensors, load sensors, electrical sensors, and bite strength sensors, the sensors monitoring at least one or more of the environment, a device within one or more of the housing assemblies, and at least one of the two or more vertebrate animals within the inner housing assembly;
tracking individuals of the two or more vertebrate animals by way of a multi object tracking software system operationally coupled to at least one optical sensor by the at least one controller, by way of at least one or more from a group of: detecting objects, reidentifying objects, generating trajectories, and aggregating features, the multi object tracking software further using at least one or more from a group of: appearance models, motion models, interaction models, exclusion models, and occlusion handling models; synchronizing tracking position of each vertebrate animal with the at least one or more sensors at least one or more of before, in real time, and after sensing an action, wherein data being captured by each at least one or more sensors is being synchronized by way of at least one time measuring device; and
assigning and storing data collected for each of the two or more vertebrate animals distinctly for that individual vertebrate animal.

11. The smart cage for housing and assaying method for multiple vertebrate animals of claim 10, including removing the at least one inner housing assembly while leaving the at least one or more sensors in place, the at least one or more sensors operationally coupled to the at least one outer housing assembly.

12. The smart cage for housing and assaying method for multiple vertebrate animals of claim 10, including measuring with at least one physiological software system the data being gathered from the at least one or more sensors and the a multi object tracking software system measures of each of the multiple vertebrate animals and determining from at least one or more from a group of: lifespan, frailty index, muscle strength, run endurance, learning and memory, balance and coordination, body weight, food intake, total time spent in sleep and awake, temporal pattern of being asleep and awake, speed of nest building, visual acuity, hearing acuity, water intake, and coat color/density, position tracking, distance traveled, movement speed, sleep time, cardiovascular health, cognition, balance and coordination, tremors, gait deficiencies, vision movement, and speed of nest building.

13. The smart cage for housing and assaying method for multiple vertebrate animals of claim 10, including tracking the two or more vertebrate animals by way of at least one RFID reader disposed on the outside cage assembly.

14. The smart cage for housing and assaying method for multiple vertebrate animals of claim 10, including integrating data from smart cage assays being generated by the at least one or more sensors, the controller transmitting data by way of at least one or more of wired and wirelessly, at least one or more of directly or by way of at least one other computer and reporting data to a central data processing system;
displaying on at least one overhead LED screen covering at least a portion of a horizontal dimension of the smart cage at least a looming spot for vision assays;
substantially continuously recording video from the cage by way of a least one near infrared camera operationally coupled to the LED screen, using the video at least for individual animal position tracking; and
illuminating with at least one or more of an infrared LED and a near infrared LED an interior portion of the inner smart cage.

15. The smart cage for housing and assaying method for multiple vertebrate animals of claim 10, including at least one or more of:
assaying learning and memory of the multiple vertebrate animals the by way of a control panel
rewarding the multiple vertebrate animals by way of at least one or more of a secondary water dispenser and a food dispenser;
assaying hearing acuity of the multiple vertebrate animals by way of at least one speaker and microphone;
assaying muscle strength of the multiple vertebrate animals by way of at least one force meter for a grip bar;
assaying bite strength of the multiple vertebrate animals by way of at least one force meter or a force sensor for a bite strength sensor; and
assaying weight of the multiple vertebrate animals by way of at least one force meter.

16. The smart cage system for housing and assaying multiple vertebrate animals of claim 10, including controlling autonomously the multiple vertebrate animals entering and leaving the smart cage to and from at least one second cage wherein the at least one second cage may be at least one or more of a second smart cage that is substantially similar to the first smart cage, a second smart cage that is different than the first smart cage, or a non-smart cage, permitting the animal, therefore, at least at some times to move to and from the smart cage to and from the other cages.

17. A smart cage system for housing and assaying multiple vertebrate animals comprising:
a plurality of inner housing assemblies and outer housing assemblies, the housing assemblies each having a top portion, a bottom portion, and at least one side portion, each inner housing assembly adapted to be at least partly disposed within and removable from the respective outer housing assembly, the housing assemblies further disposed within at least one rack assembly adapted to hold the plurality of at least one housing assemblies wherein housing assemblies may be operationally contained at least one or more of vertically and horizontally from each other, and wherein the housing assemblies may be at least partially removed from the rack independently from other housing assemblies;
the plurality of smart cages including at least one controller adapted to monitor and record data from at least one or more sensors from a group of: optical sensors, motion sensors, pressure sensors, weight sensors, temperature sensors, humidity sensors, proximity sensors, chemical sensors, volume sensors, level sensors, audio sensors, odor sensors, heartbeat sensors, brainwave sensors, body mass sensors, color sensors, rotary sensors, light sensors, oscillation sensors, balance sensors, reflex or reaction sensors, waterflow sensors, force meter sensors, load sensors, electrical sensors, and bite strength sensors, the sensors adapted to monitor at least one or more of the environment, a device within one or more of the housing assemblies, and at least one vertebrate animal within the inner housing assembly, wherein each smart cage may have unique configurations of at least one or more sensors;
the at least one or more sensors operationally synchronized at least one or more of before, in real time, and after sensing an action, wherein data captured by each at least one or more sensors can be synchronized by way of at least one time measuring device;
a multi object tracking software system operationally coupled to at least one optical sensor by the at least one controller, the multi object tracking software adapted to track individuals of the multiple vertebrate animals by way of at least one or more from a group of: object detection, object reidentification, generating trajectories, and aggregating features, the multi object tracking software further adapted to use at least one or more from a group of: appearance models, motion models, interaction models, exclusion models, and occlusion handling, the tracking of individual vertebrate animals adapted to be time synchronized; and
at least the outer housing assembly including at least one or more from a group of ports, slots, shelves, pockets, hooks, fasteners, and sleeves each adapted to retain at least one sensor.

18. The smart cage system for housing and assaying multiple vertebrate animals of claim 17, wherein the at least one or more sensors is operationally coupled to the outer housing assembly wherein the inner housing assembly may be removed without removing the at least one or more sensors.

19. The smart cage system for housing and assaying multiple vertebrate animals of claim 17, wherein at least one physiological software system is adapted, from the data gathered from the at least one or more sensors and the a multi object tracking software system measures of each of the multiple vertebrate animals from at least one or more from a group of: lifespan, frailty index, muscle strength, run endurance, learning and memory, balance and coordination, body weight, food intake, total time spent in sleep and awake, temporal pattern of being asleep and awake, speed of nest building, visual acuity, hearing acuity, water intake, coat color/density, position tracking, distance traveled, movement speed, sleep time, cardiovascular health, cognition, balance and coordination, tremors, gait deficiencies, vision movement, and speed of nest building.

20. The smart cage system for housing and assaying multiple vertebrate animals of claim 17, wherein the inner housing assembly includes a cage floor on which may be disposed at least one run wheel and a tray within the inner housing assembly containing animal feed.

21. The smart cage system for housing and assaying multiple vertebrate animals of claim 17, wherein the top portion of the outer housing assembly includes at least one or more of: a controller adapted to be a local hub to measure and integrate data associated with smart cage assays conducted using the at least one or more sensors, the controller operationally coupled at least one or more of wired and wirelessly, at least one or more of directly or by way of at least one other computer to report data to a central data processing system;
at least one overhead LED screen adapted to cover at least a portion of a horizontal dimension of the smart cage, the overhead LED screen used to display at least a looming spot for vision assays;
at least one or more of an infrared camera and a near infrared camera adapted to record video substantially continuously from the cage, the video adapted to be used at least for individual animal position tracking; and
at least one or more of an infrared LED and a near infrared LED adapted to illuminate an interior portion of the inner smart cage.

22. The smart cage system for housing and assaying multiple vertebrate animals of claim 21, wherein at least one pass filter is disposed at least one or more of in front and behind at least one infrared camera lens.

23. The smart cage system for housing and assaying multiple vertebrate animals of claim 17, wherein the side portion of the outer housing assembly includes at least one or more of: a control panel adapted for mouse learning and memory assays, at least one air valve, at least one main water dispenser and valve, at least one secondary water dispenser and valve for reward administration, at least one food dispenser and valve, at least one speaker and a microphone adapted for hearing acuity testing, at least one force meter for a grip bar for muscle strength testing, at least one force meter for weight estimation, and at least one force meter for bite strength estimation.

24. The smart cage system for housing and assaying multiple vertebrate animals of claim 17, wherein a robotic arm assembly is operationally coupled to move horizontally and vertically substantially along the entirety of the height and width of the rack assembly and is further adapted to remove housing assemblies at least partly from the rack.
